(19) United States
(12) Patent Application Publication
(10) Pub. No.: US 2025/0241786 A1
(43) Pub. Date: Jul. 31, 2025

(54) MEDICAL ELECTRIC BLANKET

(71) Applicant: GUANGDONG LAIJUN ELECTRONIC TECHNOLOGY CO., LTD., Dongguan City (CN)

(72) Inventor: Wanglai Huang, Dongguan City (CN)

(21) Appl. No.: 18/427,732

(22) Filed: Jan. 30, 2024

(30) Foreign Application Priority Data
Oct. 30, 2023 (CN) 202311423376.9

(51) Int. Cl. A61F 7/00; G05D 23/19; G05D 23/24; G08B 21/18; H05B 1/02; H05B 3/34

(52) U.S. Cl. CPC A61F 7/007; G05D 23/193; G05D 23/24; H05B 1/0202; H05B 1/0272; H05B 3/342; A61F 2007/0086; A61F 2007/0096; G08B 21/182; H05B 2203/005; H05B 2203/02

(57) ABSTRACT

A medial electric blanket includes an electric blanket body and a control host. The electric blanket body includes a first heating cable and a second heating cable thereon. The first heating cable is wound in a width direction of the electric blanket body and arranged on the electric blanket body to form a first heating zone. The first heating zone is located at middle and lower sections of the electric blanket body in a length direction of the electric blanket body. A first temperature sensor is provided in the first heating zone. The second heating cable is wound in the width direction of the electric blanket body and arranged on the electric blanket body to form a second heating zone.

### BACKGROUND OF THE INVENTION

### 1. Field of the Invention

The present invention relates to an electric blanket, and more particularly to a medical electric blanket.

### 2. Description of the Prior Art

Medical warming blankets are safe thermal insulation devices used to prevent the patient's body temperature from dropping sharply during surgery. In surgery, there are many risk factors for perioperative hypothermia, including patient factors, surgical factors, anesthetic factors, environmental factors, and the presence or absence of intervention.

The body temperature of most patients drops significantly during the perioperative period, which requires the use of external interventions. Currently, air heating is mainly used for heating. However, warm air supply systems have been phased out due to their large size interfering with surgical operations.

Existing medical electric blankets are used repeatedly after being covered with medical non-woven fabrics, which poses certain challenges to hygiene and safety.

Most of the existing disposable medical warming blankets use high-density carbon fiber heating wires sewn onto the surface of a cotton layer in a honeycomb textile pattern, and high electrically-conductive copper or silver metal wires are provided at both ends as the main conductive polar ends to achieve a more uniform temperature with minimal temperature differentials. However, the contact stability is a bit poor and the cost is high and the tensile strength is poor.

Accordingly, the inventor of the present invention has devoted himself based on his many years of practical experiences to solve these problems.

### SUMMARY OF THE INVENTION

In view of the deficiencies of the prior art mentioned above, the primary object of the present invention is to provide a medial electric blanket. The real-time operating temperatures of different heating zones of an electric blanket body can be obtained and fed back to a control host for analysis, automatic adjustment and control, so as to ensure the uniformity of temperature and safety in each zone. Besides, during the use of the electric blanket body, the temperature changes in the entire heating zone are monitored in real time.

In order to achieve the above object, the present invention adopts the following solutions.

A medial electric blanket comprises an electric blanket body and a control host for power supply and control.

The electric blanket body includes a first heating cable and a second heating cable thereon.

The first heating cable is wound in a width direction of the electric blanket body and arranged on the electric blanket body to form a first heating zone. The first heating zone is located at middle and lower sections of the electric blanket body in a length direction of the electric blanket body. A first temperature sensor is provided at a center of the first heating zone.

The second heating cable is wound in the width direction of the electric blanket body and arranged on the electric blanket body to form a second heating zone. The second heating zone is located at a periphery of the first heating zone. A second temperature sensor and a third temperature sensor are provided in the second heating zone. The second temperature sensor and the third temperature sensor are located diagonally relative to the first temperature sensor. The second temperature sensor and the third temperature sensor are not on a same side.

The first heating cable and the second heating cable are PTC (positive temperature coefficient) heating cables with NTC (negative temperature coefficient) characteristics.

The first heating cable, the second heating cable, the first temperature sensor, the second temperature sensor and the third temperature sensor are connected to the control host, respectively.

Compared with the prior art, the present invention has obvious advantages and beneficial effects as follows:

1. Through the corresponding heating cables and the corresponding temperature sensors, the real-time operating temperatures of different heating zones are obtained and fed back to the control host for analysis, automatic adjustment and control to ensure the uniformity of the temperature and safety in each zone.

2. The first and second heating cables are PTC heating cables with NTC characteristics to monitor the temperature change of the entire heating zone in real time during the use of the electric blanket body. When a foreign object such as a glove or towel covers a specific surface of the corresponding heating zone, causing localized heat dissipation and resulting in an overall NTC detection value exceeding the set value, the alarm unit will be activated immediately to improve the safety of use.

3. A self-contained on-off thermal protector is connected in series to the corresponding heating cable. When the control host fails abnormally and continues to supply power for heating, an overheat protection is activated to prevent the possibility of scalding the user in case the control host malfunctions.

4. Through the sound alarm unit and the light alarm unit, when the electric blanket body is over-temperature or in an abnormal state, the indicator light is automatically triggered and the pre-recorded audio alarm signal stored in the storage module is played to provide a warning.

### BRIEF DESCRIPTION OF THE DRAWINGS

FIG. 1 is a block diagram according to an embodiment of the present invention;

FIG. 2 is a cross-sectional view of the electric blanket body according to the embodiment of the present invention;

FIG. 3 is a top view of the electric blanket body according to the embodiment of the present invention, illustrating the first heating cable, the second heating cable, the first heating zone, the second heating zone, the first temperature sensor, the second temperature sensor and the third temperature sensor;

FIG. 4 is a circuit diagram of the power supply module according to the embodiment of the present invention;

FIG. 5 is a circuit diagram of the alarm unit according to the embodiment of the present invention;

FIG. 6 is a circuit diagram of the main control module according to the embodiment of the present invention;

FIG. 7 is a circuit diagram of the touch control module according to the embodiment of the present invention;

FIG. 8 is a circuit diagram of the first temperature sensor, the second temperature sensor and the third temperature sensor according to the embodiment of the present invention;

FIG. 9 is a circuit diagram of the storage module according to the embodiment of the present invention;

FIG. 10 is a circuit diagram of the display module according to the embodiment of the present invention; and

FIG. 11 is a circuit diagram of the output control module according to the embodiment of the present invention.

### DETAILED DESCRIPTION OF THE PREFERRED EMBODIMENTS

Embodiments of the present invention will now be described, by way of example only, with reference to the accompanying drawings.

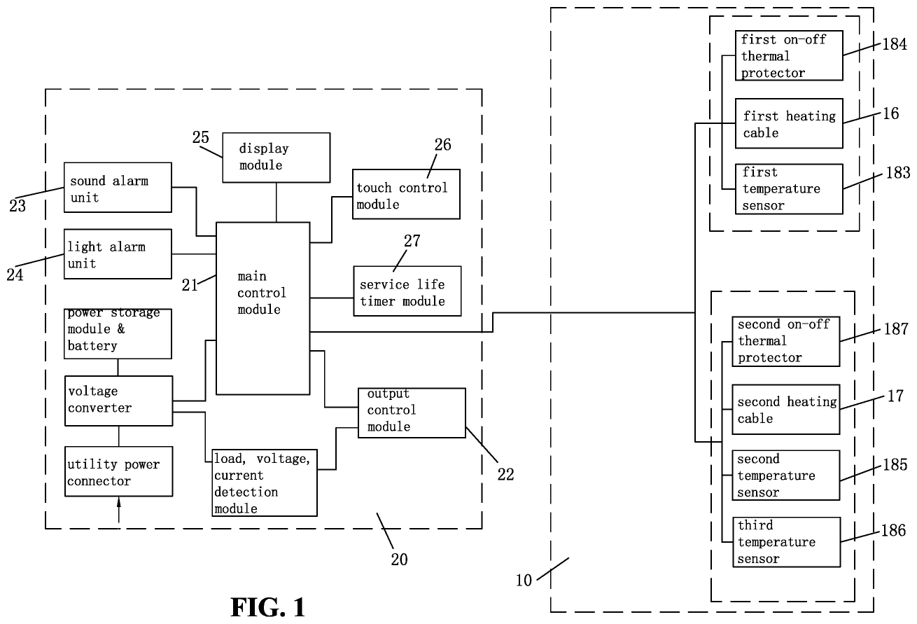

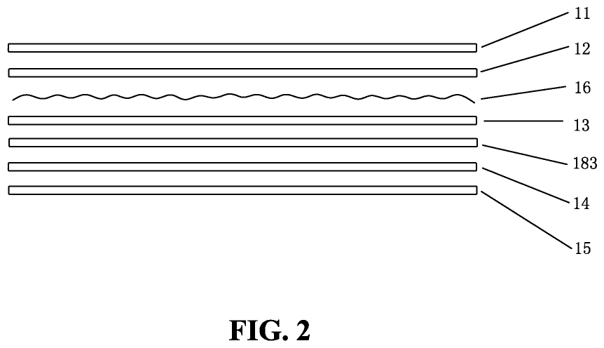

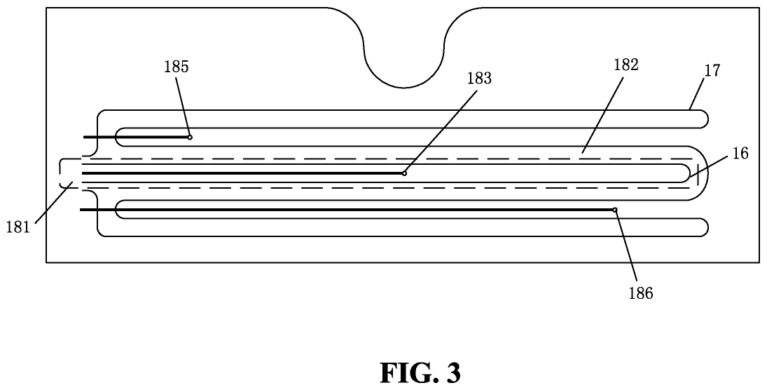

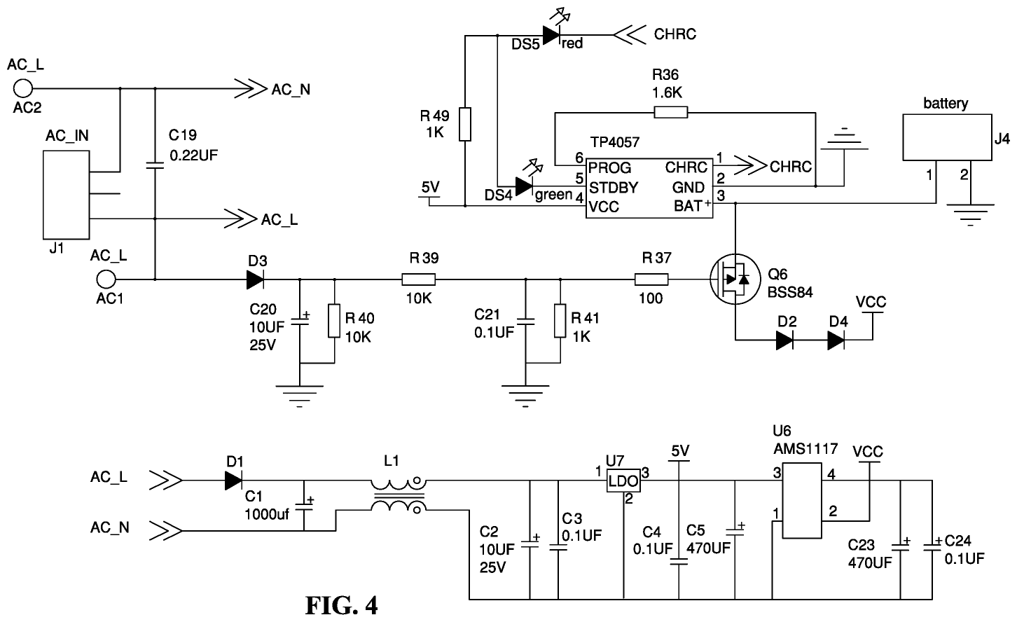

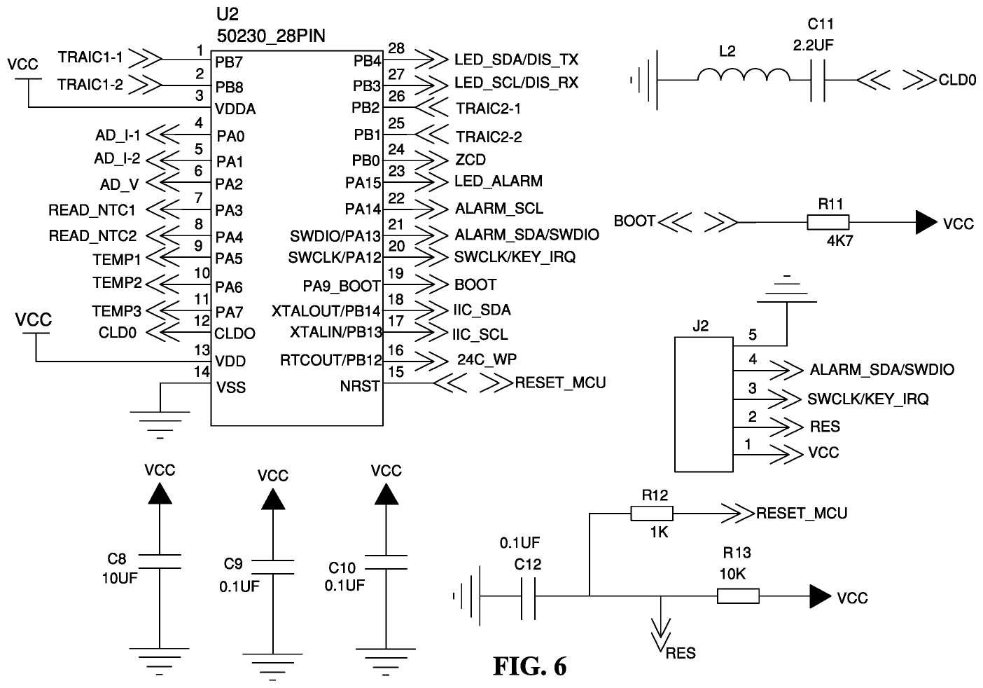

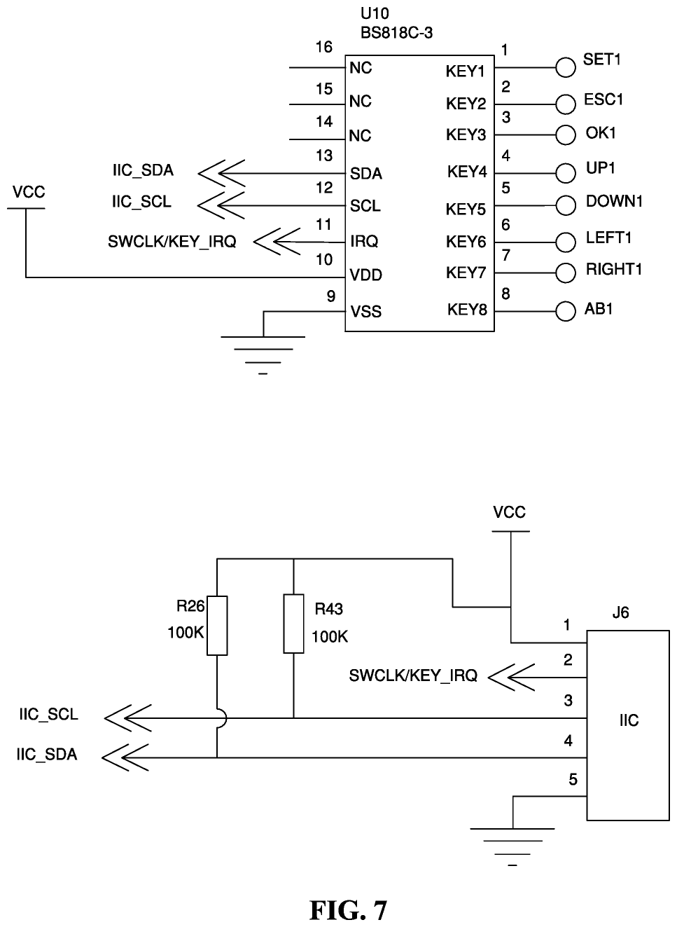

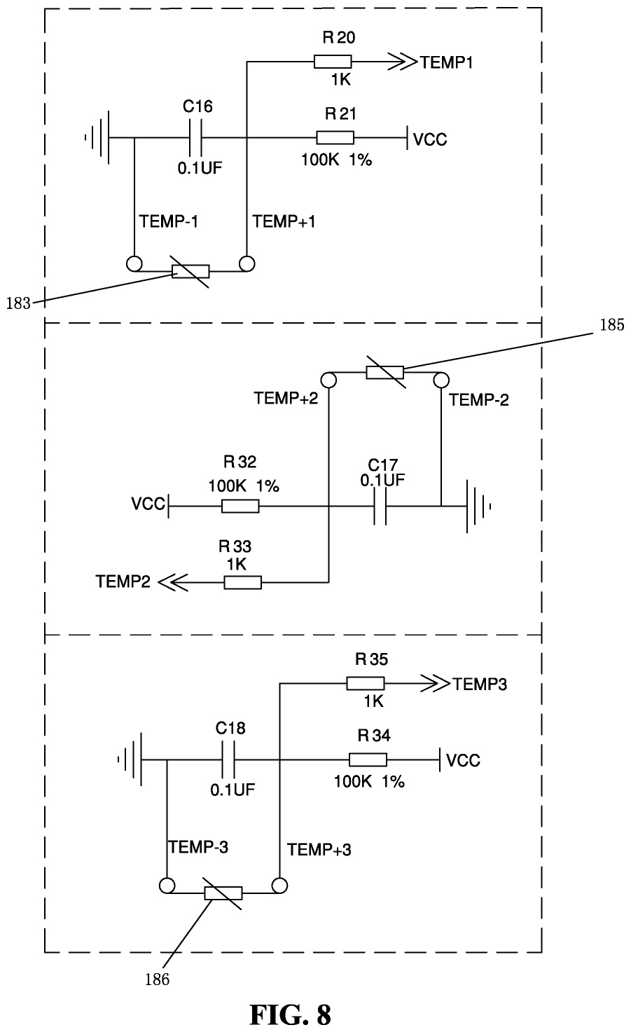

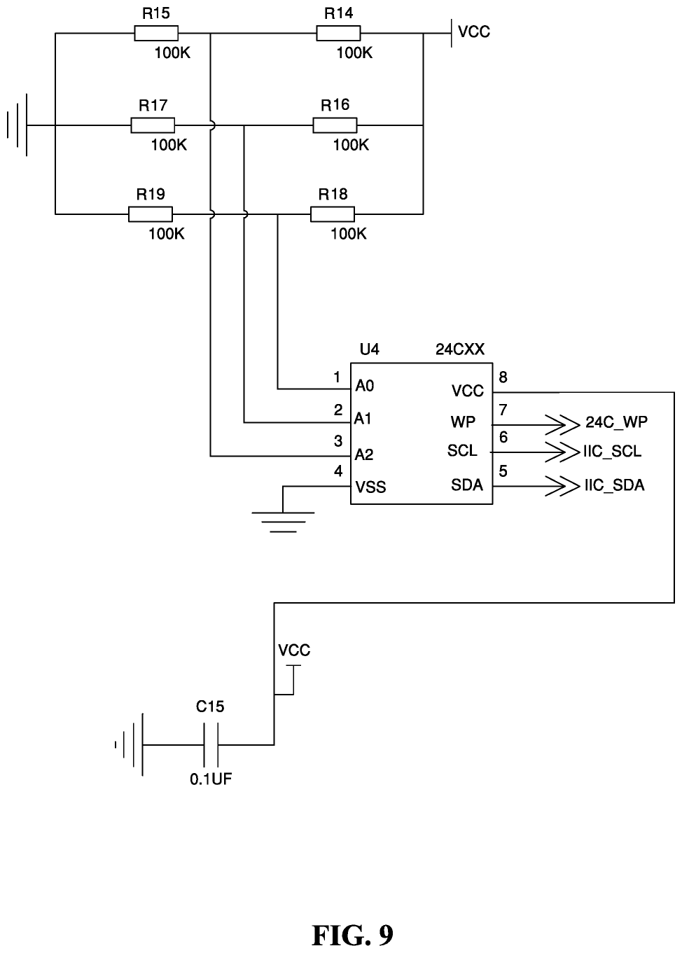

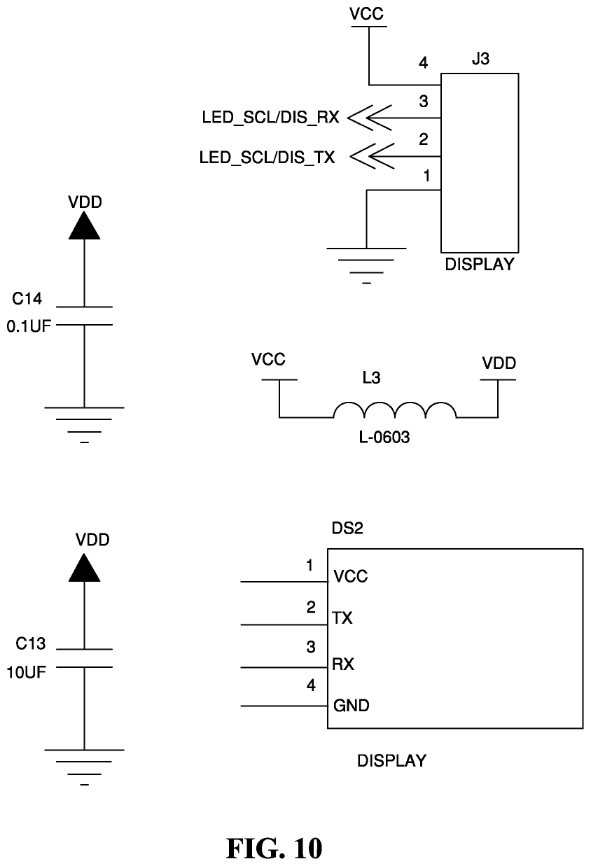

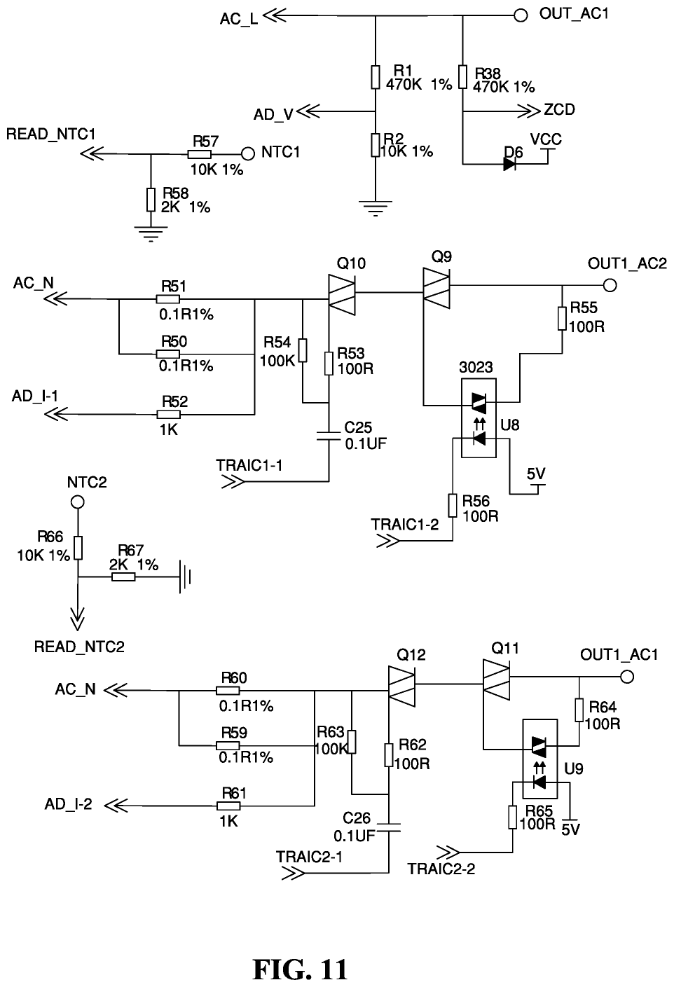

Referring to FIG. 1 through FIG. 11, the present invention discloses a medial electric blanket, comprising an electric blanket body 10 and a control host 20 for power supply and control.

In this embodiment, the electric blanket body 10 includes a top layer 11, a first spunlace non-woven fabric layer 12, a thermally conductive layer 13, a second spunlace non-woven fabric layer 14, and a bottom layer 15 used for being in contact with the human skin that are laminated in sequence from top to bottom.

Preferably, the top layer 11 is made of 30 gsm blue laminated non-woven fabric. The first spunlace nonwoven fabric layer 12 is made of 160 gsm spunlace nonwoven fabric. The second spunlace nonwoven fabric layer 14 is made of 100 gsm spunlace nonwoven fabric. The bottom layer 15 is made of 30 gsm textile non-woven fabric (SBPP).

The thermally conductive layer 13 may be a metallic copper, aluminum or non-metallic silicone thermally conductive layer. If the thermally conductive layer 13 is made of metal, the property of rapid heat conduction and diffusion of metal is utilized to achieve good diffusion of heat after being in contact with the surface of the heating cable.

In this embodiment, the product is used as a blanket to cover a person from above. Of course, the same control host may cooperate with an adjusted electric blanket body used as a heating pad for heating a person from below. This requires adjusting the materials and thickness of different layers during design and production for the heating pad to have a certain level of comfort. Generally, the thickness of the first spunlace non-woven fabric layer of the heating pad increases about 2 to 3 times the thickness of the blanket to cover a person from above, used as the material of the bottom layer to absorb shock and improve the user's experience.

The electric blanket body 10 includes a first heating cable 16 and a second heating cable 17 thereon. The first heating cable 16 and the second heating cable 17 are both located between the first spunlace non-woven fabric layer 12 and the thermally conductive layer 13.

The first heating cable 16 is wound in the width direction of the electric blanket body 10 and arranged on the electric blanket body 10 to form a first heating zone 181. The first heating zone 181 is located at the middle and lower sections of the electric blanket body in the length direction of the electric blanket body 10. A first temperature sensor 183 is provided at the center of the first heating zone 181. The first heating zone 181 has a first on-off thermal protector 184.

The second heating cable 17 is wound in the width direction of the electric blanket body 10 and arranged on the electric blanket body 10 to form a second heating zone 182. The second heating zone 182 is located at the periphery of the first heating zone 181. A second temperature sensor 185, a third temperature sensor 186 and a second on-off thermal protector 187 are provided in the second heating zone 182. The second temperature sensor 185 and the third temperature sensor 186 are located diagonally relative to the first temperature sensor 183. The second temperature sensor 185 and the third temperature sensor 186 are not on the same side.

The first temperature sensor 183, the second temperature sensor 185 and the third temperature sensor 186 are all located between the thermally conductive layer 13 and the second spunlace nonwoven layer 14.

The first heating cable 16, the second heating cable 17, the first temperature sensor 183, the second temperature sensor 185 and the third temperature sensor 186 are connected to the control host 20, respectively. The first on-off thermal protector 184 is connected in series between the first heating cable 16 and the control host 20. The second on-off thermal protector 187 is connected in series between the second heating cable 17 and the control host 20.

The first heating cable 16 and the second heating cable 17 are both PTC (positive temperature coefficient) heating cables with NTC (negative temperature coefficient) characteristics. In this embodiment, the PTC heating cable with NTC characteristics includes a PTC heating wire, an NTC temperature sensing wire, and an NTC spacer layer disposed between the PTC heating wire and the NTC temperature sensing wire. The PTC heating wire and the NTC temperature sensing wire are connected to the control host 20, respectively.

The control host 20 includes a main control module 21, an output control module 22, a sound alarm unit 23, a light alarm unit 24, a display module 25, a service life timer module 27, a touch control module 26 and a power supply module for power supply.

In this embodiment, the power supply module includes a utility power connector, a voltage converter, a power storage module, and a battery. The utility power connector is selected according to the safety standards and the sales area to be suitable for the local plug. The voltage converter has a wide-voltage design and can convert the world's normal utility power AC90V to 264V into an operating voltage, AC36V, suitable for the electric blanket body 10. The power storage module and the battery consist of a voltage regulator circuit, a charge/discharge protection circuit, and a chargeable and dischargeable lithium-ion battery cell.

Of course, in order to make this product more applicable, the operating voltage may be other voltages within the national standard voltage of DC or AC.

In this embodiment, in order to improve the demand for use, the control host 20 may be additionally equipped with a thermometer that can measure the real-time body temperature of the human body and displays it on the display module 21 in real time.

The output control module 22, the sound alarm unit 23, the light alarm unit 24, the display module 25, the touch control module 26 and the service life timer module 27 are connected to the main control module 21, respectively.

In this embodiment, the function of the service life timer module 27 is that when the control host 20 is connected to the electric blanket body 10 and each startup duration reaches a certain threshold, it defaults to a normal usage count of 1, and then accumulates the count through timing. If the startup duration does not reach a certain threshold, it will not be included in the service life time but will be counted in the total number of startups.

In this embodiment, the electric blanket body is designed for one-time use. However, as to the control host serving as an intelligent high-end display screen and touch, the cost is relatively high so the control host may be a reusable instrument. When in use, the control host is connected to the disposable electric blanket body through a connector, and its use within the safety period is controlled by means of a service life timer. Once the normal usage count reaches the preset number, the system will automatically terminate output and subsequently cease operation, thereby reducing and preventing potential malfunctions caused by the overuse of the control host.

The display module 25 is to display parameters, such as functions, operation interfaces, real-time temperature, usage count and set values, through a display screen.

The touch control module 26 is used in conjunction with the display screen for simpler operation and control by touch.

Traditional mechanical buttons for quick and auxiliary operations are provided in this embodiment, so as to provide operational control capability when touch control is not readily available.

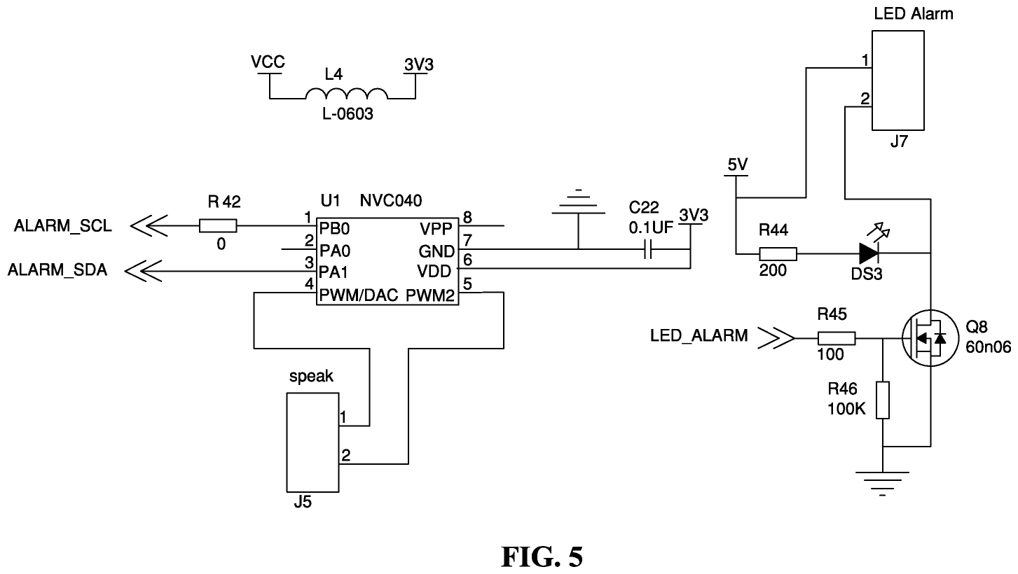

The light alarm unit 24 is configured to give a light warning in case of abnormal temperature, abnormal use or equipment abnormality. As shown in FIG. 5, the interface J7 is connected to an alarm LED.

The sound alarm unit 23 is a low-priority audio alarm using a beep, configured to give an alarm through a loudspeaker. As shown in FIG. 5, the interface J57 is connected to a loudspeaker.

The present invention further comprises a storage module connected to the main control module for playing pre-recorded audio alarm signals stored in the storage module through the sound alarm unit when the electric blanket body is over-temperature or in an abnormal state.

The output control module 22 is connected to the first heating cable 16 and the second heating cable 17. The first temperature sensor 183, the second temperature sensor 185 and the third temperature sensor 186 are connected to the main control module 21, respectively.

In this embodiment, the first temperature sensor 183, the second temperature sensor 185 and the third temperature sensor 186 use high-precision sensors with NTC characteristics, having an accuracy of up to ±0.1° C. The first temperature sensor 183, the second temperature sensor 185 and the third temperature sensor 186 are respectively arranged at the front, middle and rear sections of the electric blanket body, which solves the problem of the identification and protection of high temperatures caused by improper heat dissipation because the surface of the blanket is covered with foreign objects, meeting the standard requirements for medical electric blankets.

What is claimed is:

1. A medial electric blanket, comprising an electric blanket body and a control host for power supply and control;
the electric blanket body including a first heating cable and a second heating cable thereon;
the first heating cable being wound in a width direction of the electric blanket body and arranged on the electric blanket body to form a first heating zone, the first heating zone being located at middle and lower sections of the electric blanket body in a length direction of the electric blanket body, a first temperature sensor being provided at a center of the first heating zone;
the second heating cable being wound in the width direction of the electric blanket body and arranged on the electric blanket body to form a second heating zone, the second heating zone being located at a periphery of the first heating zone, a second temperature sensor and a third temperature sensor being provided in the second heating zone, the second temperature sensor and the third temperature sensor being located diagonally relative to the first temperature sensor, the second temperature sensor and the third temperature sensor being not on a same side;
the first heating cable and the second heating cable being PTC (positive temperature coefficient) heating cables with NTC (negative temperature coefficient) characteristics;
the first heating cable, the second heating cable, the first temperature sensor, the second temperature sensor and the third temperature sensor being connected to the control host, respectively.

2. The medial electric blanket as claimed in claim 1, wherein the PTC heating cables each include a PTC heating wire, an NTC temperature sensing wire, and an NTC spacer layer disposed between the PTC heating wire and the NTC temperature sensing wire; the PTC heating wire and the NTC temperature sensing wire are connected to the control host, respectively.

3. The medial electric blanket as claimed in claim 1, wherein the first heating zone has a first on-off thermal protector, and the first on-off thermal protector is connected in series between the first heating cable and the control host.

4. The medial electric blanket as claimed in claim 1, wherein the second heating zone has a second on-off thermal protector, and the second on-off thermal protector is connected in series between the second heating cable and the control host.

5. The medial electric blanket as claimed in claim 1, wherein the electric blanket body includes a top layer, a first spunlace non-woven fabric layer, a thermally conductive layer, a second spunlace non-woven fabric layer and a bottom layer that are laminated in sequence from top to bottom;
the first heating cable and the second heating cable are both located between the first spunlace non-woven fabric layer and the thermally conductive layer; the first temperature sensor, the second temperature sensor and the third temperature sensor are all located between the thermally conductive layer and the second spunlace nonwoven layer.

6. The medial electric blanket as claimed in claim 1, wherein the control host includes a main control module, an output control module and a power supply module for power supply, the main control module is connected to the output control module; the output control module is connected to the first heating cable and the second heating cable; the first temperature sensor, the second temperature sensor and the third temperature sensor are connected to the main control module, respectively.

7. The medial electric blanket as claimed in claim 1, wherein the control host further includes a sound alarm unit, and the sound alarm unit is connected to the main control module.

8. The medial electric blanket as claimed in claim 1, wherein the control host further includes a light alarm unit, and the light alarm unit is connected to the main control module.

9. The medial electric blanket as claimed in claim 1, wherein the control host further includes a display module, and the display module is connected to the main control module.

10. The medial electric blanket as claimed in claim 1, wherein the control host further includes a service life timer module, and the service life timer module is connected to the main control module.
